(19) United States
(12) Patent Application Publication
(10) Pub. No.: US 2022/0015660 A1
(43) Pub. Date: Jan. 20, 2022

(54) BIOELECTRICAL CANCER DIAGNOSIS OF MARGINS OF A FRESHLY DISSECTED CANCEROUS TUMOR

(71) Applicants: Mohammad Abdolahad, Tehran (IR); Reihane Mahdavi, Tehran (IR); Sajad Mehrvarz, Tehran (IR); Narges Yousefpour, Tehran (IR); Hossein Ataee, Tehran (IR); Mohammad Saeed Nikshoar, Tehran (IR); Naser Namdar Habashi, Tehran (IR); Hadi Ghafari, Tehran (IR)

(72) Inventors: Mohammad Abdolahad, Tehran (IR); Reihane Mahdavi, Tehran (IR); Sajad Mehrvarz, Tehran (IR); Narges Yousefpour, Tehran (IR); Hossein Ataee, Tehran (IR); Mohammad Saeed Nikshoar, Tehran (IR); Naser Namdar Habashi, Tehran (IR); Hadi Ghafari, Tehran (IR)

(73) Assignee: Nano Hesgarsazan Salamat Arya, Tehran (IR)

(21) Appl. No.: 17/492,252

(22) Filed: Oct. 1, 2021

Related U.S. Application Data
(60) Provisional application No. 63/087,183, filed on Oct. 3, 2020.
(60) Provisional application No. 63/105,213, filed on Oct. 24, 2020.

(51) Int. Cl. A61B 5/053; A61B 5/25; A61B 5/00

(52) U.S. Cl. CPC A61B 5/053; A61B 5/0091; A61B 5/25

(57) ABSTRACT

A method for identifying cancerous status of margins of a tumor. The method includes putting at least two electrodes of a bioimpedance sensor in contact with a target region of surface of a freshly dissected tumor tissue, measuring two impedimetric criteria associated with the target region, and detecting cancerous status of the target region based on the two measured impedimetric criteria. The two measured impedimetric criteria includes an electrical impedance magnitude of the target region at a frequency of 1 kHz (Z_{1 kHz}) and an impedance phase slope (IPS) of the target region in a frequency range of 100 kHz to 500 kHz.

### CROSS-REFERENCE TO RELATED APPLICATION

This application claims the benefit of priority from pending U.S. Provisional Patent Application Ser. No. 63/087,183 filed on Oct. 3, 2020, and entitled “BIOELECTRICAL PATHOLOGY OF THE BREAST” and pending U.S. Provisional Patent Application Ser. No. 63/105,213 filed on Oct. 24, 2020, and entitled “BIOPSY-FREE CANCER DIAGNOSTIC NEEDLE FOR REAL-TIME DISTINGUISHMENT OF BENIGN AND MALIGNANT BREAST MASSES WITH BI-RADS AND PATHOLOGICAL CALIBRATIONS”, which are both incorporated herein by reference in their entirety.

### TECHNICAL FIELD

The present disclosure generally relates to cancer diagnosis, and particularly, to real-time diagnosis of cancerous margins of a dissected tumor from a cancer patient utilizing electrical impedance spectroscopy of a dissected tumor margins.

### BACKGROUND

There is a worldwide requirement for new methodologies for precise and complete scanning of a dissected cancerous tumor margins during a tumor removal surgery to not only prevent cancer recurrences by removing cancerous lesions from surgical boundaries but also to conserve healthy parts of tissues and organs, and minimize impact on a patient's lifestyle. Frozen-section of a dissected tumor is a conventional method in intraoperative margin diagnostics. But fast freezing of a tissue leads to some misdiagnoses due to undesirable cellular staining and microscopic cell transformations. Therefore, making an accurate diagnosis from frozen sections is difficult and shows at least 15-20% of misdiagnosis. Another concern in tumor margin detection by intraoperative frozen-section is imperfect freezing of fatty tissues which results in opaque hematoxylin-eosin (H&E) staining and hence misdiagnosis. So a presence of fatty lesions in tumor margin would perturb a frozen sample preparation. Thus, in non-advanced medical centers or some developing countries, lack of well-experienced pathologists can be a big challenge. On the other hand, most of frozen-section samples are prepared from margins of dissected tumors which not only do not included observable or palpable cancerous masses but also may include distributed lesions and foci of pre-malignant and in-situ cancer lesions. Solid tumor masses contain large numbers of cancerous or suspicious cells which make a cancer diagnosis easy for a pathologist via sampling only one part of a tissue suspected to be cancerous. Whereas, a number of malignant or high-risk cells are so rare in tumor margins which affect accuracy in margin diagnosis due to a limitation of numbers of samples that can be drawn from a region suspected to be cancerous in a pathological assay.

One of the technologies developed to determine a cancerous state of body tissues is impedance analysis. Under an alternating electrical excitation, biological tissues exhibit a complex electrical impedance that depends on tissue composition, structure, health status, and physiological or pathological properties. Therefore, normal and malignant tissues have different impedimetric parameters due to their different frequency-dependent dielectric relaxation and electric current blocking abilities. Several reports have been published on impedance analysis of dissected human tumor masses while only a few researchers focused on tumor margin analysis with clinical diagnosis. It might be due to the complicated distinction between the dielectric behaviors of normal tissues (such as stroma or fat) after being infiltrated by the rare distribution of cancer cells. No investigation has been reported on pathologically categorized responses of an impedance analyzer for cancer diagnosis to be useful in intraoperative clinical evaluation of tumor margins with acceptable accuracy.

Hence, there is a need for devices, systems, and methods for scoring clearance or malignancy involvement/presence of a dissected tumor margins. Furthermore, there is a need for fast, cost-effective, and simple scanning whole margins of a dissected tumor to identify cancer involved margins; thereby, removing any remaining cancerous margins within a patient's body. Moreover, there is a need for reducing or even terminating multiple sampling from a cancer patient for doing multiple inaccurate pathological tests.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes a method for identifying cancerous status of margins of a tumor. The method may include putting two electrodes of a bioimpedance sensor in contact with a target region of a surface of a freshly dissected tumor tissue, measuring two impedimetric criteria associated with the target region, and detecting a cancerous status of the target region based on the two measured impedimetric criteria.

In an exemplary implementation, measuring two impedimetric criteria associated with the target region may include measuring an electrical impedance magnitude of the target region at a frequency of 1 kHz (Z_{1 kHz}) and measuring impedance phase slope (IPS) of the target region in a frequency range of 100 kHz to 500 kHz. In an exemplary implementation, detecting the cancerous status of the target region may include determining the target region is a benign region if the measured Z_{1 kHz} is less than a first reference impedance value and the measured IPS is more than a first reference IPS, determining the target region is a cancerous region if the measured Z_{1 kHz} is less than the first reference impedance value and the measured IPS is less than a second reference IPS, and determining the target region is a fatty region if the measured Z_{1 kHz} is more than a second reference impedance value. In an exemplary embodiment, the second reference IPS may be equal to the first reference IPS or less.

In an exemplary implementation, measuring the two impedimetric criteria associated with the target region may include connecting the two electrodes of the bioimpedance sensor to an impedance analyzer device, applying an alternating current (AC) voltage in a sweeping range of frequencies to the two electrodes, measuring an impedance magnitude of an electrical impedance value of the target region at frequency of 1 kHz (Z_{1 kHz}), measuring a set of electrical impedance phase values respective to the swept range of frequencies between 100 kHz and 500 kHz, and calculating the IPS respective to the swept range of frequencies between 100 kHz and 500 kHz. In an exemplary embodiment, the sweeping range of frequencies may include a frequency range between 1 kHz and 500 kHz.

In an exemplary implementation, applying the AC voltage in the sweeping range of frequencies to the two electrodes may include applying an AC voltage with an amplitude of 0.4 V in the sweeping range of frequencies to the two electrodes.

In an exemplary implementation, calculating the IPS may include calculating a slope of the measured set of electrical impedance phase values versus the swept range of frequencies. The IPS may be defined by:

$$IPS = \frac{{Phase}_{2} - {Phase}_{1}}{log ( {Frequency}_{2} ) - log ( {Frequency}_{1} )}$$

Where, Phase₁ may be a first impedance phase value measured at a first frequency value (Frequency₁) of 100 kHz and Phase₂ may be a second impedance phase value measured at a second frequency value (Frequency₂) of 500 kHz.

In an exemplary embodiment, the freshly dissected tumor tissue may include a tumor tissue dissected less than 30 minutes from a human or animal, where a time period up to 30 minutes may be passed after dissection of the tumor tissue. In an exemplary embodiment, the target region may include a part of surface of the freshly dissected tumor tissue with an area of 4 mm² and a depth of 2 mm.

In an exemplary implementation, putting the two electrodes of the bioimpedance sensor in contact with the target region may include putting two respective distal ends of the two electrodes on the surface of the freshly dissected tumor tissue at the target region. In an exemplary implementation, putting the two electrodes of the bioimpedance sensor in contact with the target region may further include forming a uniform pressurized contact between the respective distal ends of the two electrodes and the target region by applying a vacuum suction pressure throughout the two electrodes. In an exemplary implementation, applying the vacuum suction pressure throughout the two electrodes may include connecting a vacuum pump to respective proximal ends of the two electrodes utilizing a tubular line and applying a vacuum pressure of at least 20 KPa to the respective proximal ends of the two electrodes utilizing the vacuum pump.

In an exemplary embodiment, the freshly dissected tumor tissue may include a freshly dissected breast tumor. In such implementation, detecting the cancerous status of the target region may include determining the target region is a benign breast region if the measured Z_{1 kHz} is less than 2.5 kΩ and the measured IPS is more than 0.3, determining the target region is a cancerous breast region if the measured Z_{1 kHz} is less than 2.5 kΩ and the measured IPS is negative (less than zero), and determining the target region is a fatty breast region if the measured Z_{1 kHz} is more than 4.8 kΩ.

In an exemplary implementation, detecting the cancerous status of the target region may further include determining exemplary target region is a benign breast region having clusters of cancerous breast cells if the measured Z_{1 kHz} is less than 2.5 kΩ and the measured IPS is between zero and 0.3. In another exemplary implementation, detecting the cancerous status of the target region may further include determining the target region is a benign fatty breast region including a plurality of fatty breast cells if a range for the measured Z_{1 kHz} and the measured IPS includes at least one of the measured Z_{1 kHz} is between 2.5 kΩ and 3.5 kΩ and the measured IPS is between −1 and 2, and the measured Z_{1 kHz} is between 3.5 kΩ and 4.8 kΩ and the measured IPS is between −2 and 1. In an additional exemplary implementation, detecting the cancerous status of the target region may further include determining the target region is a fatty breast region having clusters of cancerous breast cells if a range for the measured Z_{1 kHz} and the measured IPS includes at least one of the measured Z_{1 kHz} is between 2.5 kΩ and 3.5 kΩ and the measured IPS is between −4 and −1, and the measured Z_{1 kHz} is between 3.5 kΩ and 4.8 kΩ and the measured IPS is between −5 and −2.

In another general aspect of the present disclosure, a system for identifying cancerous status of margins of a tumor is disclosed. In an exemplary embodiment, the system may include a bioimpedance sensor, an impedance analyzer device, and a processing unit electrically connected to the impedance analyzer device. In an exemplary embodiment, the bioimpedance sensor may include at least two tubular electrodes. In an exemplary embodiment, each respective electrode of the two tubular electrodes may include an electrically conductive hollow rod. In an exemplary embodiment, each respective electrode may include a distal end and a proximal end, where each respective distal end may be configured to be put in contact with a target region of surface of a tumor tissue dissected less than 30 minutes from a human or an animal, and each respective proximal end may be configured to be connected to the impedance analyzer device. In an exemplary embodiment, the impedance analyzer device may be connected to the bioimpedance sensor, where respective proximal ends of the at least two tubular electrodes may be in connection with the impedance analyzer device via at least one of an electrical connector and a wireless connection.

In an exemplary embodiment, the processing unit may include a memory having processor-readable instructions stored therein and a processor. In an exemplary embodiment, the processor may be configured to access the memory and execute the processor-readable instructions. In an exemplary embodiment, the processor may be configured to perform a method by executing the processor-readable instructions. In an exemplary embodiment, the method may include applying an alternating current (AC) voltage in a sweeping range of frequencies between 1 kHz and 500 kHz to the at least two tubular electrodes utilizing the impedance analyzer device, measuring an electrical impedance value of the target region at frequency of 1 kHz (Z_{1 kHz}) utilizing the impedance analyzer device, measuring a set of electrical impedance phase values respective to the swept range of frequencies between 100 kHz and 500 kHz utilizing the impedance analyzer device, calculating impedance phase slope (IPS) respective to the swept range of frequencies between 100 kHz and 500 kHz, and detecting cancerous status of the target region based on the measured Z_{1 kHz} and the calculated IPS.

In an exemplary implementation, detecting the cancerous status of the target region based on the measured Z_{1 kHz} and the calculated IPS may include determining the target region is a benign region if the measured Z_{1 kHz} is less than a first reference impedance value and the measured IPS is more than a first reference IPS, determining the target region is a cancerous region if the measured Z_{1 kHz} is less than the first reference impedance value and the measured IPS is less than a second reference IPS, and determining the target region is a fatty region if the measured Z_{1 kHz} is more than a second reference impedance value. In an exemplary embodiment, the second reference IPS may be equal to the first reference IPS or less.

In an exemplary embodiment, the dissected tumor tissue may include a dissected breast tumor. In such implementation, detecting the cancerous status of the target region may include determining the target region is a benign breast region if the measured Z_{1 kHz} is less than 2.5 kΩ and the measured IPS is more than 0.3, determining the target region is a cancerous breast region if the measured Z_{1 kHz} is less than 2.5 kΩ and the measured IPS is negative (less than zero), and determining the target region is a fatty breast region if the measured Z_{1 kHz} is more than 4.8 kΩ.

In an exemplary implementation, detecting the cancerous status of the target region may further include determining exemplary target region is a benign breast region having clusters of cancerous breast cells if the measured Z_{1 kHz} is less than 2.5 kΩ and the measured IPS is between zero and 0.3. In another exemplary implementation, detecting the cancerous status of the target region may further include determining the target region is a benign fatty breast region including a plurality of fatty breast cells if a range for the measured Z_{1 kHz} and the measured IPS includes at least one of the measured Z_{1 kHz} is between 2.5 kΩ and 3.5 kΩ and the measured IPS is between −1 and 2, and the measured Z_{1 kHz} is between 3.5 kΩ and 4.8 kΩ and the measured IPS is between −2 and 1. In an additional exemplary implementation, detecting the cancerous status of the target region may further include determining the target region is a fatty breast region having clusters of cancerous breast cells if a range for the measured Z_{1 kHz} and the measured IPS includes at least one of the measured Z_{1 kHz} is between 2.5 kΩ and 3.5 kΩ and the measured IPS is between −4 and −1, and the measured Z_{1 kHz} is between 3.5 kΩ and 4.8 kΩ and the measured IPS is between −5 and −2.

In an exemplary implementation, calculating the IPS may include calculating a slope of the measured set of electrical impedance phase values versus the swept range of frequencies defined by:

$$IPS = \frac{{Phase}_{2} - {Phase}_{1}}{log ( {Frequency}_{2} ) - log ( {Frequency}_{1} )}$$

Where, Phase₁ may be a measured impedance phase value at a first frequency value (Frequency₁) of 100 kHz and Phase₂ may be a measured impedance phase value at a second frequency value (Frequency₂) of 500 kHz.

In an exemplary embodiment, the system may further include a vacuum pump. In an exemplary embodiment, the vacuum pump may be configured to be connected to the respective proximal ends of the at least two tubular electrodes utilizing a tubular line and be electrically connected to the processing unit. In an exemplary embodiment, the method may further include forming a uniform connection between distal ends of the at least two tubular electrodes and the target region by applying, utilizing the vacuum pump, a vacuum pressure of at least 20 KPa to the respective proximal ends of the at least two tubular electrodes.

In an exemplary embodiment, each respective electrode of the two tubular electrodes may include a stainless steel hollow rod with a length between 10 mm and 20 mm and an internal diameter between 0.5 mm and 2 mm. In an exemplary embodiment, an electrically insulating layer with a thickness between 0.5 mm and 1 mm may be placed around parts of each respective electrode of the two tubular electrodes.

In an exemplary embodiment, the bioimpedance sensor may further include an electrode holder, a handle, and a cap. In an exemplary embodiment, the electrode holder may include at least two hollow openings, where each hollow opening of the at least two hollow openings may encompass a middle part of each electrode of the at least two tubular electrodes. In an exemplary embodiment, the middle part of each electrode may include a respective part of each electrode except the respective distal end and the proximal end. In an exemplary embodiment, the handle may include a tubular member. In an exemplary embodiment, the handle may include a distal end and a proximal end, where the electrode holder may be fixed inside the distal end of the handle. In an exemplary embodiment, the handle may be configured to facilitate utilizing the at least two tubular electrodes. In an exemplary embodiment, the handle may be configured to facilitate putting the respective distal ends of the at least two tubular electrodes with the target region, facilitate applying a vacuum pressure through the at least two tubular electrodes, and contain an electrical wire connecting the impedance analyzer device to the respective proximal ends of the at least two tubular electrodes. In an exemplary embodiment, the cap may be configured to seal the proximal end of the handle by fastening the cap around the proximal end of the handle. In an exemplary embodiment, the cap may include two openings, including a first opening that may be configured to pass the electrical wire there through, where the electrical wire may be connected to the impedance analyzer device and a second opening that may be configured to connect to a vacuum pump by fastening a flexible tubular line around the second opening, where the flexible tubular line may be connected to the vacuum pump. In an exemplary embodiment, each two respective openings of the at least two hollow openings embedded on the electrode holder may have a distance between 2 mm and 5 mm

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1 illustrates an exemplary system for identifying cancerous status of margins of a tumor, consistent with one or more exemplary embodiments of the present disclosure.

FIG. 2 illustrates an exemplary schematic view of an exemplary implementation of an exemplary bioimpedance sensor, consistent with one or more exemplary embodiments of the present disclosure.

FIG. 3A illustrates an exemplary method for identifying cancerous status of margins of a tumor, consistent with one or more exemplary embodiments of the present disclosure.

FIG. 3B illustrates an exemplary implementation of measuring two impedimetric criteria associated with an exemplary target region, consistent with one or more exemplary embodiments of the present disclosure.

FIG. 3C illustrates an exemplary implementation of detecting cancerous status of an exemplary target region, consistent with one or more exemplary embodiments of the present disclosure.

FIG. 4A illustrates an exemplary schematic cross-section view of an exemplary implementation of putting exemplary two tubular electrodes of an exemplary bioimpedance sensor in contact with an exemplary target region of surface of an exemplary freshly dissected tumor tissue, consistent with one or more exemplary embodiments of the present disclosure.

FIG. 4B shows another schematic cross-section view of an exemplary implementation of putting two exemplary tubular electrodes of an exemplary bioimpedance sensor in contact with an exemplary target region of surface of an exemplary freshly dissected tumor tissue, consistent with one or more exemplary embodiments of the present disclosure.

FIG. 5 illustrates an example computer system in which an embodiment of the present disclosure, or portions thereof, may be implemented as computer-readable code, consistent with one or more exemplary embodiments of the present disclosure.

FIG. 6 illustrates an image of an exemplary head part of an exemplary fabricated bioimpedance sensor, consistent with one or more exemplary embodiments of the present disclosure.

FIG. 7A illustrates impedance magnitude and phase diagrams of an exemplary normal muscle and an exemplary tumor tissue of mice recorded by an exemplary system for identifying cancerous status of a tumor, consistent with one or more exemplary embodiments of the present disclosure.

FIG. 7B illustrates exemplary hematoxylin-eosin (H&E) assays of exemplary mice healthy muscular tissue and exemplary mice malignant tissue respective to recorded impedance magnitude and phase diagrams of FIG. 7A, consistent with one or more exemplary embodiments of the present disclosure.

FIG. 8 illustrates impedance spectroscopy of exemplary normal and cancerous regions tested from 10 mice models with their related Z_{1 kHz} and impedance phase slope (IPS) in frequencies between 100 kHz and 500 kHz and H&E assays, consistent with one or more exemplary embodiments of the present disclosure.

FIG. 9 illustrates impedance magnitude and phase diagrams for different types of tissues (with pathologically distinct patterns) based on measured Z_{1 kHz} and IPS, consistent with one or more exemplary embodiments of the present disclosure.

FIG. 10 illustrates six types of breast tissues and their respective impedance spectroscopy classification parameters with an example for each type, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein, an exemplary system is disclosed for measuring electrical properties of a dissected tissue. An exemplary dissected tissue may include a dissected tumor from a cancer patient. An exemplary system may be described here for measuring electrical impedance of margins of a dissected tumor from an animal body or a human body. An exemplary system for measuring electrical impedance of margins of a dissected tumor may be utilized for identifying cancerous status of tumor margins of a freshly dissected tumor.

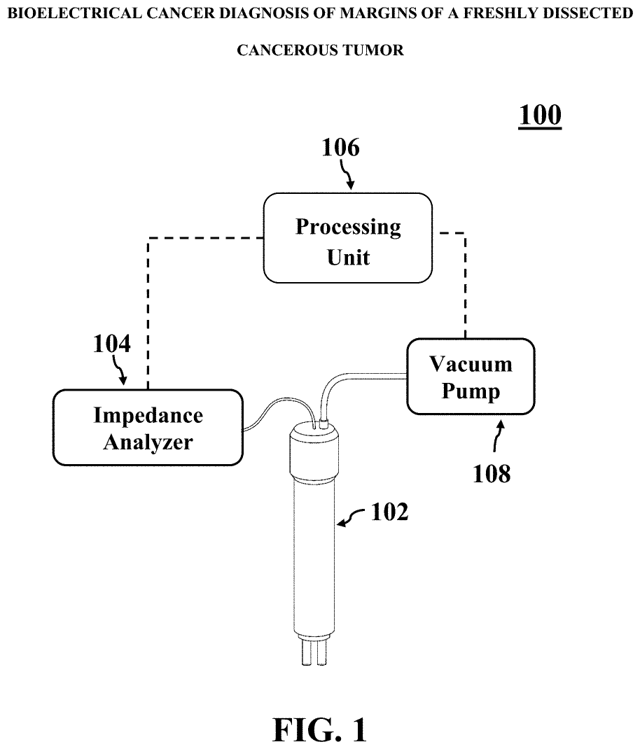

FIG. 1 shows exemplary system 100 for identifying cancerous status of margins of a tumor, consistent with one or more exemplary embodiments of the present disclosure. In an exemplary embodiment, system 100 may include exemplary bioimpedance sensor 102, exemplary impedance analyzer device 104, and exemplary processing unit 106 electrically connected to impedance analyzer device 104. In an exemplary embodiment, bioimpedance sensor 102 may be electrically connected to impedance analyzer device 104. In an exemplary embodiment, system 100 may further include exemplary vacuum pump 108, where vacuum pump 108 may be electrically connected to processing unit 106. In an exemplary embodiment, impedance analyzer device 104 may include an impedance meter device.

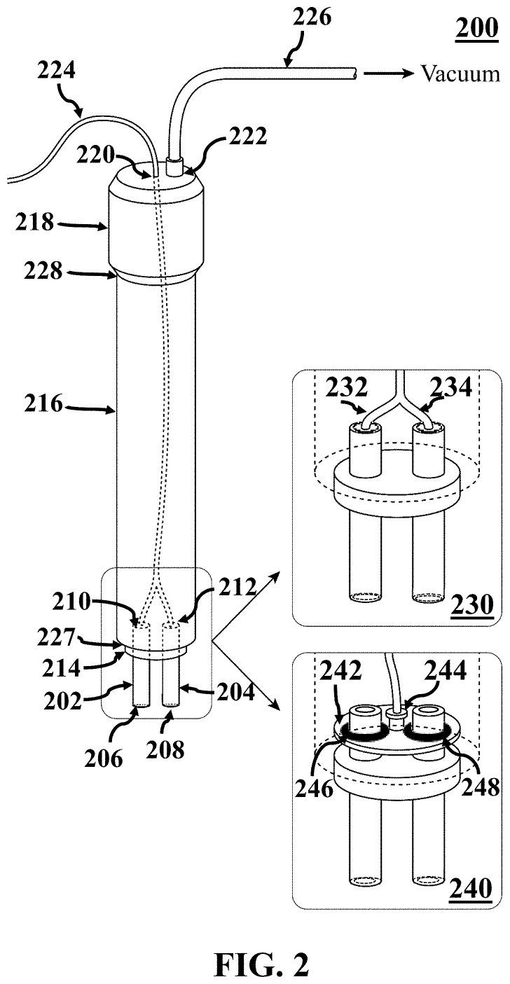

FIG. 2 shows a schematic view of exemplary implementation 200 of bioimpedance sensor 102, consistent with one or more exemplary embodiments of the present disclosure. In an exemplary embodiment, exemplary bioimpedance sensor 102 may include at least two tubular electrodes 202 and 204. In an exemplary embodiment, exemplary bioimpedance sensor 102 may further include exemplary electrode holder 214, exemplary handle 216, and exemplary cap 218.

In an exemplary embodiment, each respective electrode of two tubular electrodes 202 and 204 may include respective distal end 206 and 208 and respective proximal end 210 and 212. In an exemplary embodiment, each of respective distal ends 206 and 208 may be configured to be put in contact with a target region of surface of a freshly dissected tumor tissue. In an exemplary embodiment, each of respective proximal ends 210 and 212 may be configured to be connected to impedance analyzer device 104.

In an exemplary embodiment, each respective electrode of two tubular electrodes 202 and 204 may include an electrically conductive hollow rod. In an exemplary embodiment, each respective electrode of two tubular electrodes 202 and 204 may include a hollow rod made of a biocompatible electrically conductive material, for example, stainless steel. In an exemplary embodiment, each respective electrode of two tubular electrodes 202 and 204 may include a biomedical or biocompatible needle-shaped tubes. In an exemplary embodiment, each respective electrode of two tubular electrodes 202 and 204 may have a length between about 10 mm and about 20 mm and an internal diameter between about 0.5 mm and about 2 mm.

In an exemplary embodiment, each respective electrode of two tubular electrodes 202 and 204 may be covered with a layer of an electrically insulating material, for example, a layer of plastic. In an exemplary embodiment, a layer of an electrical insulating thermal varnish may be adhered around parts of two tubular electrodes 202 and 204 except respective distal ends 206 and 208. In an exemplary embodiment, the layer of the electrically insulating material may prevent direct contact between two tubular electrodes 202 and 204 with each other; thereby, resulting in preventing electrical noises in an exemplary electrical impedance measurement utilizing two tubular electrodes 202 and 204. In an exemplary embodiment, the layer of the electrically insulating material may provide a constant surface area being in contact between two tubular electrodes 202 and 204 and a target region to be determined whether is cancerous or not. In an exemplary embodiment, the layer of the electrically insulating material may have a thickness between about 0.5 mm and about 1 mm. In an exemplary embodiment, the layer of the electrically insulating material may be placed or coated or adhered around each respective electrode of the two tubular electrodes 202 and 204 except around a part of two tubular electrodes 202 and 204 at respective distal ends 206 and 208. In an exemplary embodiment, distal ends 206 and 208 of respective two tubular electrodes 202 and 204 may remain uncoated-allowing for putting an electrical contact between distal ends 206 and 208 and a target region of surface of a freshly dissected tumor tissue in an electrical measurement, for example, measuring an electrical impedance of a target region of surface of a freshly dissected tumor tissue. In an exemplary embodiment, a length of about 0.01 mm to about 2 mm of each of two tubular electrodes 202 and 204 from distal ends 206 and 208 may remain uncoated configured to be put in direct contact with an exemplary target region of surface of a freshly dissected tumor tissue. In an exemplary embodiment, a length of about 0.1 mm to about 2 mm of each of two tubular electrodes 202 and 204 from distal ends 206 and 208 may remain uncoated configured to be put in direct contact with an exemplary target region of surface of a freshly dissected tumor tissue. In an exemplary embodiment, only a cross section of distal ends 206 and 208 of respective two tubular electrodes 202 and 204 may remain uncoated configured to be put in direct contact with an exemplary target region of surface of a freshly dissected tumor tissue.

In an exemplary embodiment, two tubular electrodes 202 and 204 may be placed and fixed inside two respective hollow openings of electrode holder 214. In an exemplary embodiment, electrode holder 214 may be a device configured to hold respective electrodes and may include at least two hollow openings, where each respective hollow opening may have a diameter equal to an outer diameter of each of two tubular electrodes 202 and 204. In an exemplary embodiment, each of two tubular electrodes 202 and 204 may be placed and sealed inside each respective hollow opening of electrode holder 214. In an exemplary embodiment, each respective hollow opening of the at least two hollow openings may encompass a middle part of each respective electrode of two tubular electrodes 202 and 204. In an exemplary embodiment, the middle part of each respective electrode of two tubular electrodes 202 and 204 may include a respective part of each electrode except respective distal ends 206 and 208 and proximal ends 210 and 212. In an exemplary embodiment, each two respective openings of the at least two hollow openings electrode holder 214 may have a distance from each other in a range between about 2 mm and about 5 mm.

In an exemplary embodiment, bioimpedance sensor 200 may further include exemplary handle 216. In an exemplary embodiment, handle 216 may include a tubular member or a cylindrical member. In an exemplary embodiment, handle 216 may include distal end 227 and proximal end 228. In an exemplary embodiment, electrode holder 214 encompassing middle parts of two tubular electrodes 202 and 204 may be placed and fixed distal end 227.

In an exemplary embodiment, a hollow space between electrode holder 214 and wall of handle 216 at distal end 227 may be sealed so that only two distal ends 206 and 208 may remain as open ends of bioimpedance sensor 200 in a case of applying a vacuum pressure through two tubular electrodes 202 and 204 utilizing vacuum pump 108. In an exemplary embodiment, a hollow space between electrode holder 214 and wall of handle 216 at distal end 227 may be sealed in a case where a vacuum pressure through two tubular electrodes 202 and 204 utilizing vacuum pump 108 may be applied for forming an intense and reinforced contact between two distal ends 206 and 208 and an exemplary target region.

In an exemplary embodiment, handle 216 may be configured to facilitate utilizing two tubular electrodes 202 and 204, facilitate putting respective distal ends 206 and 208 of two tubular electrodes 202 and 204 with a target region, facilitate applying a vacuum pressure through two tubular electrodes 202 and 204, and contain electrical wire 224 connecting impedance analyzer device 104 to respective proximal ends 210 and 212 of two tubular electrodes 202 and 204.

In an exemplary embodiment, bioimpedance sensor 102 may further include cap 218. In an exemplary embodiment, cap 218 may be configured to seal proximal end 228 of handle 216 by fastening cap 218 around proximal end 228. In an exemplary embodiment, proximal end 228 may include an externally threaded end of handle 216 and cap 218 may have an internally threaded end. In an exemplary embodiment, the cap 218 may be fastened around proximal end 228 by screwing internally threaded end of cap 218 around externally threaded proximal end 228 of handle 216. In an exemplary embodiment, cap 218 may include two openings 220 and 222, including first opening 220 and second opening 222. In an exemplary embodiment, first opening 220 may be configured to pass electrical wire 224 there through; allowing for connecting two tubular electrodes 202 and 204 to impedance analyzer device 104. In an exemplary embodiment, second opening 222 may be configured to connect to vacuum pump 108 by fastening or fixing a first end of exemplary flexible tubular line 226 around second opening 222. In an exemplary embodiment, a second end of flexible tubular line 226 may be connected to vacuum pump 108.

In an exemplary embodiment, proximal ends 210 and 212 of respective two tubular electrodes 202 and 204 may be connected to impedance analyzer device 104 by utilizing at least one of electrical wire 224 and a wireless connection. FIG. 2 shows two exemplary implementations 230 and 240 for connecting electrical wire 224 to proximal ends 210 and 212. In exemplary implementation 230, electrical wire 224 may include two electrical wires 232 and 234 respectively soldered to proximal ends 210 and 212. In exemplary implementation 230, two electrical wires 232 and 234 may be insulated from each other via a respective layer of an electrically insulating material coated around parts of each of electrical wires 232 and 234. In an exemplary embodiment, a first end of each of two electrical wires 232 and 234 may be connected to a respective proximal end of proximal ends 210 and 212. In an exemplary embodiment, a second end of electrical wire 232 may be connected to a ground output of impedance analyzer device 104 and a second end of electrical wire 234 may be connected to a alternating stimulating voltage signal of impedance analyzer device 104.

In exemplary implementation 240, electrical wire 224 may include two electrical wires (not illustrated) similar to electrical wires 232 and 234. In an exemplary implementation, exemplary two electrical wires may be electrically insulated from each other and soldered to respective proximal ends 210 and 212 similar to electrical wires 232 and 234. In an exemplary implementation, a first end of exemplary two electrical wires may be connected respectively to two tubular electrodes 202 and 204 via an interface electrical connector 244 placed on exemplary substrate 242. In an exemplary embodiment, electrical connector 244 may include a two-pin electrical connector that may be configured to connect exemplary two respective electrical wires to two respective tubular electrodes 202 and 204. In an exemplary embodiment, substrate 242 may have two openings, where each of proximal ends 210 and 212 may pass and fixed through a respective opening of the two openings of substrate 242. In an exemplary embodiment, electrical connector 244 may be attached to substrate 242. In an exemplary embodiment, two electrically conductive ring-shaped elements 246 and 248 may be attached on substrate 242 so that two electrically conductive ring-shaped elements 246 and 248 being embedded around two tubular electrodes 202 and 204 and while two electrically conductive ring-shaped elements 246 and 248 being in contact with electrical connector 244. In an exemplary embodiment, two electrically conductive ring-shaped elements 246 and 248 may include two copper rings. In an exemplary embodiment, a respective second end of one of exemplary two electrical wires may be connected to a ground output of impedance analyzer device 104 and a respective second end of the other electrical wire of exemplary two electrical wires may be connected to a sinusoidal stimulating voltage output of impedance analyzer device 104.

In an exemplary embodiment, substrate 242 may include a support printed circuit board (PCB) with at least two copper tracks thereon. Exemplary at least two copper tracks may form electrically conductive elements similar to electrically conductive ring-shaped elements 246 and 248. Exemplary at least two copper tracks may provide two respective electrically conductive paths between each of two tubular electrodes 202 and 204 and electrical connector 244; allowing for separately connecting each of two tubular electrodes 202 and 204 to impedance analyzer device 104.

In another exemplary implementation, a Bluetooth device or a Bluetooth module may be attached to substrate 242 and two electrically conductive ring-shaped elements 246 and 248; allowing for a wireless connection between two tubular electrodes 202 and 204 and impedance analyzer device 104. The wireless connection may allow for simplifying utilizing bioimpedance sensor 102 by removing redundant wires that may require to sanitize iteratively, etc. in medical applications.

In an exemplary embodiment, processing unit 106 may include a memory having processor-readable instructions stored therein and a processor. The processor may be configured to access the memory and execute the processor-readable instructions. In an exemplary implementation, the processor may perform a method by executing the processor-readable instructions, for example, a method for identifying cancerous status of margins of a tumor described herein below.

In an exemplary implementation, an exemplary method for identifying cancerous status of margins of a tumor may be disclosed here. In an exemplary implementation, a cancerous tumor dissected from a cancer patient's body may be investigated to determine whether cancerous margins remain in a cancer patient's body or not. In an exemplary implementation of the present disclosure, an exemplary method for detecting cancerous status of margins of a freshly dissected tumor utilizing exemplary system 100 is disclosed.

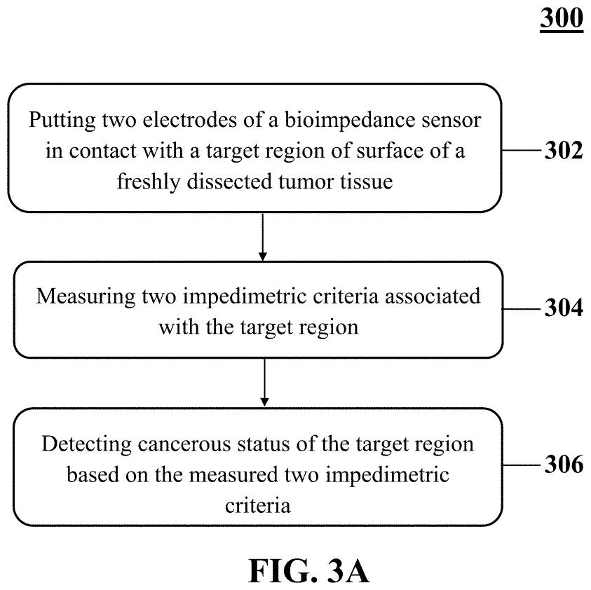

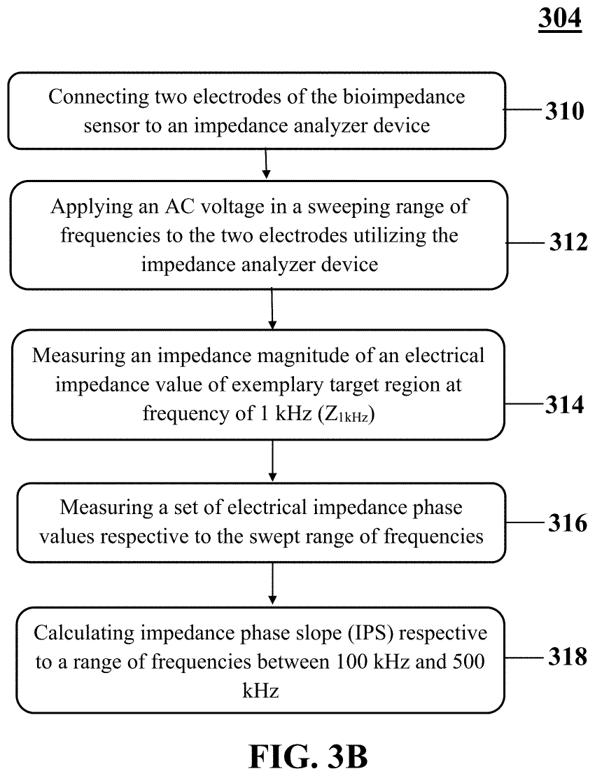

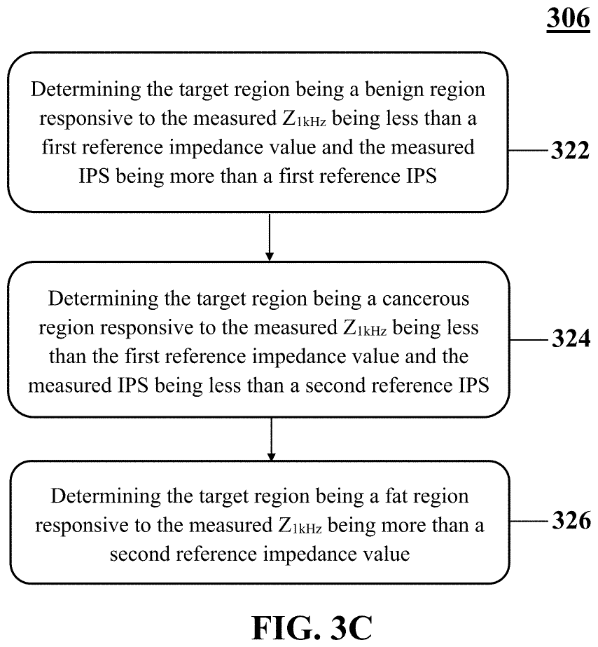

FIG. 3A shows exemplary method 300 for identifying cancerous status of margins of a tumor, consistent with one or more exemplary embodiments of the present disclosure. In an exemplary implementation, method 300 may include putting at least two electrodes of a bioimpedance sensor in contact with a target region of surface of a freshly dissected tumor tissue (step 302), measuring two impedimetric criteria associated with the target region (step 304), and detecting cancerous status of the target region based on the measured two impedimetric criteria (step 306).

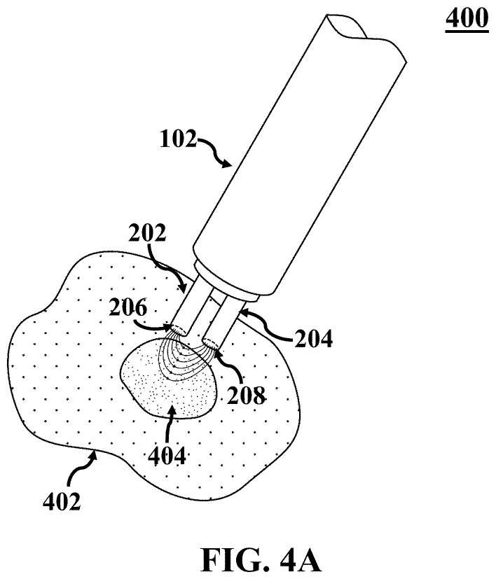

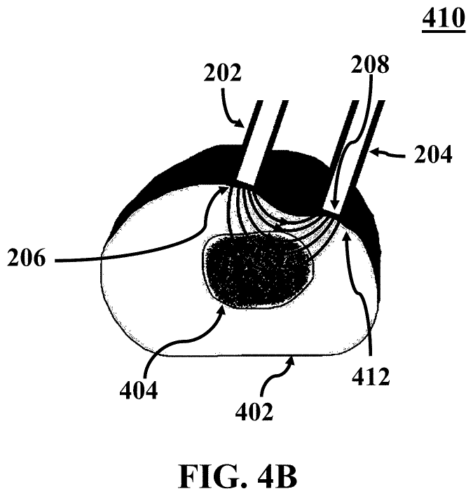

In detail, step 302 may include putting at least two electrodes of a bioimpedance sensor in contact with a target region of surface of a freshly dissected tumor tissue. In an exemplary implementation, putting at least two electrodes of a bioimpedance sensor in contact with a target region of surface of a freshly dissected tumor tissue may include putting two tubular electrodes 202 and 204 of exemplary bioimpedance sensor 102 in contact with a target region of surface of a freshly dissected tumor tissue. FIG. 4A shows a schematic cross-section view of an exemplary implementation 400 of putting two tubular electrodes 202 and 204 of exemplary bioimpedance sensor 102 in contact with an exemplary target region 404 of surface of an exemplary freshly dissected tumor tissue 402 (step 302), consistent with one or more exemplary embodiments of the present disclosure. In an exemplary implementation, putting two tubular electrodes 202 and 204 of exemplary bioimpedance sensor 102 in contact with exemplary target region 404 of surface of exemplary freshly dissected tumor tissue 402 (step 302) may include putting two respective distal ends 206 and 208 of two tubular electrodes 202 and 204 on surface of exemplary freshly dissected tumor tissue 402 at exemplary target region 404.

In an exemplary implementation, putting two tubular electrodes 202 and 204 of exemplary bioimpedance sensor 102 in contact with exemplary target region 404 of surface of exemplary freshly dissected tumor tissue 402 (step 302) may further include generating a uniform connection between respective distal ends 206 and 208 of two tubular electrodes 202 and 204 and exemplary target region 404 by applying a vacuum pressure to respective proximal ends 210 and 212 of two tubular electrodes 202 and 204 utilizing vacuum pump 108. In an exemplary implementation, generating the uniform connection between two distal ends 206 and 208 and exemplary target region 404 may include uniforming and reinforcing a pressurized connection between two distal ends 206 and 208 and exemplary target region 404. In an exemplary implementation, generating the uniform connection between two distal ends 206 and 208 and exemplary target region 404 may lead to decrease an electrical contact impedance between two distal ends 206 and 208 and exemplary target region 404 and conduct precise electrical measurements. In an exemplary implementation, generating the uniform connection between two distal ends 206 and 208 and exemplary target region 404 may include forming a soft reproducible electrical contact between two distal ends 206 and 208 and exemplary target region 404 by uniforming and intensifying a constant contact between two distal ends 206 and 208 and exemplary target region 404 utilizing vacuum pump 108. In an exemplary embodiment, vacuum pump 108 may include a surgical suction pump with a suction pressure of more than about 20 KPa vacuum pressure corresponding to a suction flow rate of more than about 20 lit/min. In an exemplary implementation, vacuum pump 108 may include a surgical suction pump with a suction pressure of more than about 60 KPa vacuum pressure or a surgical suction pump with a suction flow rate of about 90 lit/min or more. In an exemplary embodiment, vacuum pump 108 may include a rotary pump with a maximum suction pressure of about 0.01 Torr.

In an exemplary implementation, reinforcing the connection between two distal ends 206 and 208 and exemplary target region 404 may include connecting vacuum pump 108 to respective proximal ends 210 and 212 of two tubular electrodes 202 and 204 utilizing tubular line 226 and applying a vacuum suction pressure throughout two tubular electrodes 202 and 204 utilizing vacuum pump 108. In an exemplary implementation, applying the vacuum suction pressure throughout two tubular electrodes 202 and 204 may include applying a vacuum pressure of at least 0.01 Torr to respective proximal ends 210 and 212 utilizing vacuum pump 108. In an exemplary implementation, applying the vacuum suction pressure throughout two tubular electrodes 202 and 204 may include applying a vacuum pressure of at least 20 KPa to respective proximal ends 210 and 212 utilizing vacuum pump 108. In an exemplary implementation, applying the vacuum suction pressure throughout two tubular electrodes 202 and 204 utilizing vacuum pump 108 may be carried out by processing unit 106 electrically connected to vacuum pump 108.

FIG. 4B shows another schematic cross-section view of an exemplary implementation 410 of putting two tubular electrodes 202 and 204 of exemplary bioimpedance sensor 102 in contact with exemplary target region 404 of surface of an exemplary freshly dissected tumor tissue 402 (step 302), consistent with one or more exemplary embodiments of the present disclosure. In an exemplary implementation, a cross section of two distal ends 206 and 208 may be stuck to exemplary marginal region 412 of exemplary freshly dissected tumor tissue 402 for electrical measurements from exemplary target region 404, for example, measuring two impedimetric criteria associated with exemplary target region 404 (step 304).

In an exemplary embodiment, exemplary freshly dissected tumor tissue 402 may include a cancerous tumor mass which may be freshly dissected from a human or animal. In an exemplary embodiment, exemplary freshly dissected tumor tissue 402 may include a cancerous tumor mass which its dissection time may have not passed more than a few minutes. In an exemplary embodiment, exemplary freshly dissected tumor tissue 402 may include a cancerous tumor mass where a time period up to about 30 minutes may be passed after dissection of the cancerous tumor mass. In an exemplary embodiment, the cancerous tumor mass may include all types of cancerous tumors in animals or humans' bodies. In an exemplary embodiment, the cancerous tumor mass may include a breast tumor, a malignant breast tumor, a liver tumor, a colon tumor, a prostate tumor, a bladder tumor, a thyroid tumor, an invasive ductal carcinoma (IDC) tumor, a soft tissue sarcoma tumor, such as Leiomyosarcoma and Spindle cell sarcoma, etc.

In an exemplary embodiment, target region 404 may include a portion of tumor margins of exemplary freshly dissected tumor tissue 402. In an exemplary embodiment, exemplary target region 404 of surface of exemplary freshly dissected tumor tissue 402 may include a part of surface of freshly dissected tumor tissue 402 with an area of about 4 mm² and a depth of about 2 mm. In an exemplary implementation, exemplary method 300 may be performed for a plurality of exemplary target regions 404 of exemplary freshly dissected tumor tissue 402 up to cover identifying cancerous status of all surface area of exemplary freshly dissected tumor tissue 402. Thereafter, identifying cancerous status of all margins over surface of exemplary freshly dissected tumor tissue 402 may be achieved.

Moreover, referring to FIG. 3A, step 304 may include measuring two impedimetric criteria associated with exemplary target region 404 utilizing impedance analyzer device 104. In an exemplary implementation, measuring two impedimetric criteria associated with exemplary target region 404 (step 304) may include measuring an electrical impedance magnitude of exemplary target region 404 at a frequency of about 1 kHz (Z_{1 kHz}) and measuring impedance phase slope (IPS) of exemplary target region 404 in a frequency range of about 100 kHz to about 500 kHz. In an exemplary implementation, measuring two impedimetric criteria associated with exemplary target region 404 (step 304) may be done during a time period in a range between about 2 seconds and about 10 seconds. In an exemplary implementation, measuring two impedimetric criteria associated with exemplary target region 404 (step 304) may be done during a time period of about 5 seconds.

In an exemplary implementation, measuring two impedimetric criteria associated with exemplary target region 404 in step 304 may be done via an electrochemical impedance spectroscopy (EIS) approach. The EIS approach may include applying a known voltage or current as an electrical stimulus to a biological material (e.g., target region 404 where respective distal ends 206 and 208 of two tubular electrodes 202 and 204 may be placed there) and measuring a resulting current or voltage as a response. In an exemplary embodiment, two tubular electrodes 202 and 204 put in contact with target region 404 may be configured to act as impedance stimulation and measurement electrodes. The biological material may produce a complex electrical impedance in response to the electrical stimulus. The complex electrical impedance may depend on the biological material's composition, structures, health status, and physiological or pathological properties. The EIS approach may involve measuring at least one of electrical impedance Z, admittance Y, impedance modulus |Z|, the permittivity, and combinations thereof as a function of frequency to characterize the biological material. The biological material may conduct an electric current and hence may have an associated impedance parameter. The biological material may include cells and extracellular medium. The cells may be made of cell membrane and intracellular medium. Both extracellular and intracellular medium may include ionic solutions that may be electrically resistive. The cell membrane may be made of a lipid bilayer and proteins and may be primarily capacitive. An electrical impedance associated with this capacitance may be dependent on frequency.

FIG. 3B shows an exemplary implementation of measuring two impedimetric criteria associated with exemplary target region 404 (step 304), consistent with one or more exemplary embodiments of the present disclosure. In an exemplary implementation, measuring two impedimetric criteria associated with exemplary target region 404 (step 304) may include connecting two tubular electrodes 202 and 204 of bioimpedance sensor 102 to impedance analyzer device 104 (step 310), applying an alternating current (AC) voltage in a sweeping range of frequencies to two tubular electrodes 202 and 204 utilizing impedance analyzer device 104 (step 312), measuring an impedance magnitude of an electrical impedance value Z of exemplary target region 404 at a pre-determined frequency X (Z_{X}, i.e., Z_{1 kHz}) (step 314), measuring a set of electrical impedance phase values respective to the swept range of frequencies (step 316), and calculating the IPS respective to a range of frequencies between about 100 kHz and about 500 kHz (step 318).

In an exemplary implementation, connecting two tubular electrodes 202 and 204 of bioimpedance sensor 102 to impedance analyzer device 104 (step 310) may be done by connecting electrical wire 224 to impedance analyzer device 104. In an exemplary implementation, electrical wire 224 may be attached to proximal ends 210 and 212 of respective two tubular electrodes 202 and 204 via one of implementations 230 or 240.

In an exemplary implementation, applying an AC voltage in a sweeping range of frequencies to two tubular electrodes 202 and 204 utilizing impedance analyzer device 104 (step 312) may include applying an AC voltage in a sweeping range of frequencies in a range between about 1 Hz and about 1 MHz to two tubular electrodes 202 and 204. In an exemplary implementation, applying an AC voltage in a sweeping range of frequencies to two tubular electrodes 202 and 204 utilizing impedance analyzer device 104 may include applying an AC voltage in a sweeping range of frequencies in a range between about 1 kHz and about 500 kHz to two tubular electrodes 202 and 204. In an exemplary implementation, applying an AC voltage in a sweeping range of frequencies to two tubular electrodes 202 and 204 utilizing impedance analyzer device 104 may include applying an AC voltage with an amplitude in a range between about 0.2 V and about 0.8 V in the sweeping range of frequencies to two tubular electrodes 202 and 204. In an exemplary implementation, applying an AC voltage in a sweeping range of frequencies to two tubular electrodes 202 and 204 utilizing impedance analyzer device 104 may include applying an AC voltage with an amplitude of about 0.4 V in the sweeping range of frequencies to two tubular electrodes 202 and 204. In an exemplary implementation, applying a constant AC voltage may be laterally applied on exemplary target region 404 and an electric current may be established through exemplary target region 404 as shown in FIGS. 4A and 4B. In an exemplary embodiment, impedance analyzer device 104 may include an impedance meter device that may be configured to apply a constant voltage/current alternating signal between two tubular electrodes 202 and 204; thereby, generating an electric field between two tubular electrodes 202 and 204. In an exemplary embodiment, impedance analyzer device 104 may be configured to measure and record an electrical current/voltage signal generated between two tubular electrodes 202 and 204. Then, an impedance magnitude may be calculated by dividing an electrical voltage to an electrical amplitude, and phase shift in electrical current signal against voltage signal may be considered as an impedance phase in each signal frequency. In an exemplary implementation, impedance analyzer device 104 may include a customized precision impedance meter that may be designed and fabricated to work in a constant voltage mode. It means that an alternative signal with constant voltage (about 0.4 V amplitude) may be applied between two tubular electrodes 202 and 204 in step 312. Such electrical stimulation may be equal to an electric field of about 200 V/m. Then, a phase shift in current signal may be measured in next steps.

In an exemplary implementation, step 314 may include measuring an impedance magnitude of an electrical impedance value Z of exemplary target region 404 at a pre-determined frequency X, therefore step 314 may include measuring Z_{X}. In an exemplary implementation, step 314 may include measuring an impedance magnitude of an electrical impedance value Z of exemplary target region 404 at frequency of about 1 kHz (Z_{1 kHz}). In an exemplary implementation, Z_{1 kHz} may include an impedance magnitude of an electrical impedance value measured at an applied frequency of about 1 kHz utilizing impedance analyzer device 104.

Furthermore, step 316 may include measuring a set of electrical impedance phase values respective to the swept range of frequencies. In an exemplary implementation, a respective set of electrical impedance phase values may be measured at each frequency of the swept range of frequencies. In an exemplary implementation, step 316 may further include plotting the measured set of electrical impedance phase values versus the swept range of frequencies.

In an exemplary implementation, step 318 may include calculating the IPS respective to a range of frequencies between about 100 kHz and about 500 kHz based on the plotted set of electrical impedance phase values versus the swept range of frequencies. In an exemplary implementation, calculating the IPS may include calculating a slope of an exemplary plotted and/or measured set of electrical impedance phase values within an interval between two determined frequencies of the swept range of frequencies. In an exemplary implementation, calculating the IPS may include calculating a slope of an exemplary plotted and/or measured set of electrical impedance phase values within an interval between a first frequency (Frequency₁) and a second frequency (Frequency₂) using Equation (1) as follows:

$$IPS = \frac{{Phase}_{2} - {Phase}_{1}}{log ( {Frequency}_{2} ) - log ( {Frequency}_{1} )} \qquad Equation ( 1 )$$

In Equation (1), Phase₂ may be a second impedance phase value measured at frequency value of Frequency₂ and Phase₁ may be a first impedance phase value measured at frequency value of Frequency₁. In an exemplary implementation, Frequency₁, Frequency₂, Phase₁, and Phase₂ may be read and extracted from an exemplary measured set of electrical impedance phase values in step 316 and the respective swept range of frequencies and/or an exemplary plotted impedance phase diagram in step 316. In an exemplary implementation, Frequency₁ may be a frequency of 100 kHz and Frequency₂ may be a frequency of 500 kHz. In an exemplary implementation, Phase₂ is a measured impedance phase value at frequency value of 500 kHz (Frequency₂) and Phase₁ is a measured impedance phase value at frequency value of 100 kHz (Frequency₁).

Furthermore, referring to FIG. 3A, step 306 may include detecting cancerous status of exemplary target region 404 based on the measured two impedimetric criteria (the measured Z_{1 kHz} and the calculated IPS). FIG. 3C shows an exemplary implementation of detecting cancerous status of exemplary target region 404 (step 306), consistent with one or more exemplary embodiments of the present disclosure.

In an exemplary implementation, detecting cancerous status of exemplary target region 404 (step 306) may include determining the target region is a benign region if the measured Z_{X} (e.g., Z_{1 kHz}) is less than a first reference impedance value and the measured IPS is more than a first reference IPS (step 322), determining the target region is a cancerous region if the measured Z_{X} (e.g., Z_{1 kHz}) is less than the first reference impedance value and the measured IPS is less than a second reference IPS (step 324), and determining the target region is a fatty region if the measured Z_{X} (e.g., Z_{1 kHz}) is more than a second reference impedance value (step 326). In an exemplary embodiment, the second reference IPS may be equal to the first reference IPS or less.

In an exemplary embodiment, if exemplary freshly dissected tumor tissue 402 is dissected from a tissue or organ having no fatty cells, such as thyroid, colon, cervix, etc., detecting cancerous status of exemplary target region 404 (step 306) may include determining the target region is a benign region if the measured Z_{X} (e.g., Z_{1 kHz}) is less than the first reference impedance value and the measured IPS is more than the first reference IPS (step 322) and determining the target region is a cancerous region if the measured Z_{X} (e.g., Z_{1 kHz}) is less than the first reference impedance value and the measured IPS is less than the second reference IPS (step 324).

In an exemplary embodiment, if exemplary freshly dissected tumor tissue 402 is dissected from a tissue or organ having fatty cells in structure thereof, such as breast, detecting cancerous status of exemplary target region 404 (step 306) may include determining the target region is a benign region if the measured Z_{X} (e.g., Z_{1 kHz}) is less than the first reference impedance value and the measured IPS is more than the first reference IPS (step 322), determining the target region is a cancerous region if the measured Z_{X} (e.g., Z_{1 kHz}) is less than the first reference impedance value and the measured IPS is less than the second reference IPS (step 324), and determining the target region is a fatty region if the measured Z_{X} (e.g., Z_{1 kHz}) is more than the second reference impedance value (step 326). In an exemplary embodiment, if the measured Z_{X} (e.g., Z_{1 kHz}) is between the first reference impedance value and the second reference impedance value, exemplary target region 404 may include a mixture (combination) of fatty cells and tissue that may include cancer cells or not.

In an exemplary embodiment, exemplary freshly dissected tumor tissue 402 may include a freshly dissected breast tumor. In an exemplary embodiment, for an exemplary freshly dissected breast tumor, the first reference impedance value may be equal to about 2.5 kΩ, the second reference impedance value may be equal to about 4.8 kΩ, the first reference IPS may be equal to about 0.3, and the second reference IPS may be equal to zero. In an exemplary implementation, detecting cancerous status of exemplary target region 404 may include determining the target region is a benign breast region if the measured Z_{1 kHz} is less than about 2.5 kΩ and the measured IPS is more than about 0.3 (step 322), determining the target region is a cancerous breast region if the measured Z_{1 kHz} is less than about 2.5 kΩ and the measured IPS is negative (less than zero), and determining the target region is a fatty breast region if the measured Z_{1 kHz} is more than about 4.8 kΩ.

In an exemplary implementation, detecting cancerous status of exemplary target region 404 may further include determining exemplary target region 404 is a benign breast region having clusters of cancerous breast cells if the measured Z_{1 kHz} is less than about 2.5 kΩ and the measured IPS is between zero and about 0.3. In another exemplary implementation, detecting cancerous status of exemplary target region 404 may further include determining exemplary target region 404 is a benign fatty breast region including a plurality of fatty breast cells if a range for the measured Z_{1 kHz} and the measured IPS includes at least one of the measured Z_{1 kHz} is between about 2.5 kΩ and about 3.5 kΩ and the measured IPS is between about −1 and about 2, and the measured Z_{1 kHz} is between about 3.5 kΩ and about 4.8 kΩ and the measured IPS is between about −2 and about 1. In an additional exemplary implementation, detecting cancerous status of exemplary target region 404 may further include determining exemplary target region 404 is a fatty breast region having clusters of cancerous breast cells if a range for the measured Z_{1 kHz} and the measured IPS includes at least one of the measured Z_{1 kHz} is between about 2.5 kΩ and about 3.5 kΩ and the measured IPS is between about −4 and about −1, and the measured Z_{1 kHz} is between about 3.5 kΩ and about 4.8 kΩ and the measured IPS is between about −5 and about −2.

In an exemplary implementation, steps 304 and 306 of exemplary method 300 may be carried out by processing unit 106 utilizing bioimpedance sensor 102, impedance analyzer device 104, and vacuum pump 108. In an exemplary implementation, processing unit 106 may include a memory having processor-readable instructions stored therein and a processor. The processor may be configured to access the memory and execute the processor-readable instructions.

In an exemplary implementation, the processor may be configured to perform a method by executing the processor-readable instructions. In an exemplary implementation, the method may include conducting steps 304 and 306 of exemplary method 300. In an exemplary implementation, the method may include measuring two impedimetric criteria associated with the target region (step 304) and detecting cancerous status of the target region based on the measured two impedimetric criteria (step 306).

In an exemplary implementation, the processor may be further configured to record and communicate the measured Z_{1 kHz}, the calculated IPS, the measured set of electrical impedance phase values from exemplary target region 404 of surface of exemplary freshly dissected tumor tissue 402, and detected cancerous status of exemplary target region 404 to an individual or an expert who may utilize processing unit 106.

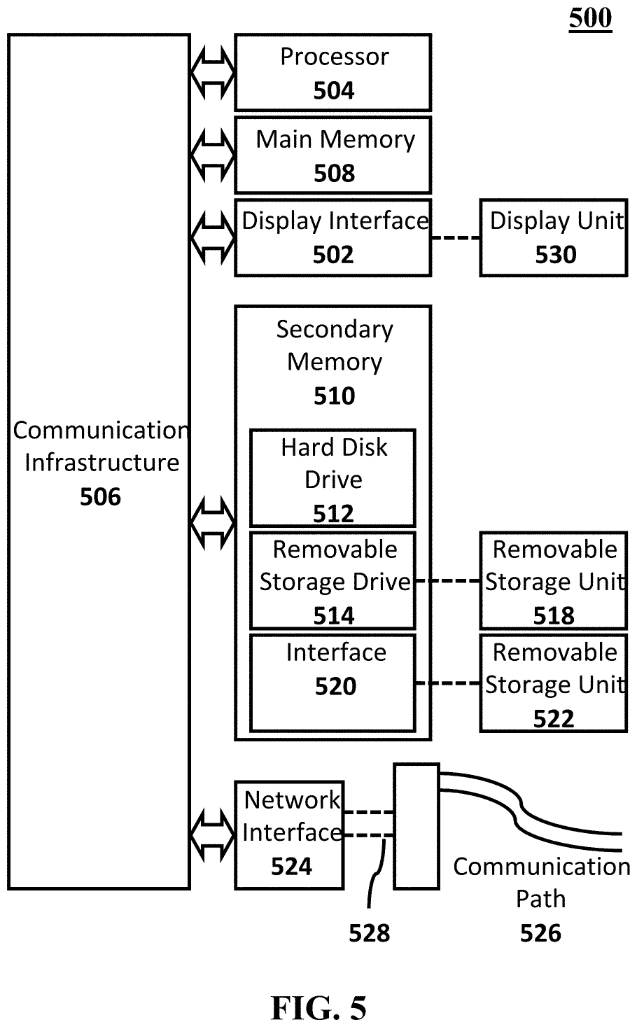

FIG. 5 shows an example computer system 500 in which an embodiment of the present disclosure, or portions thereof, may be implemented as computer-readable code, consistent with one or more exemplary embodiments of the present disclosure. For example, computer system 500 may include an example of processing unit 106, and steps 304 and 306 of exemplary flowchart 300 presented in FIG. 3A, may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIG. 1, FIG. 2, and FIG. 3A.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the present disclosure is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative implementations, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 300 illustrated by FIG. 3A, discussed above. Accordingly, such computer programs represent controllers of computer system 500. Where an exemplary embodiment of method 300 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Fabrication of a Bioimpedance Sensor

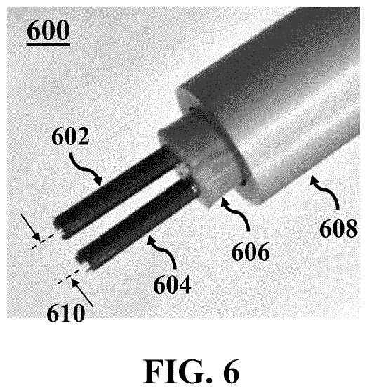

In this example, an exemplary bioimpedance sensor similar to bioimpedance sensor 102 was designed and fabricated. FIG. 6 shows an image of an exemplary head part of an exemplary fabricated bioimpedance sensor 600, consistent with one or more exemplary embodiments of the present disclosure. Exemplary fabricated bioimpedance sensor 600 includes two electrodes 602 and 604, which include two medical-grade stainless steel Veterinary Hypodermic G14 needles that were cut and polished to make 15 mm long needle tubes 602 and 604. An outer and an inner diameter of the needle tubes 602 and 604 are about 2 mm and about 1 mm, respectively. Needle tubes 602 and 604 were shielded by two plastic covers (with black color in FIG. 6), so that only the electrodes' cross surface may be in contact with a tumor tissue. Needle tubes 602 and 604 were embedded in exemplary electrode holder 606 with distance 610 of about 4 mm, and also needle tubes 602 and 604 were soldered to a support printed circuit board (PCB) (not observable in FIG. 6) similar to substrate 242. The PCB was placed in exemplary sealed cover 608 similar to handle 216 that makes it easy to apply vacuum through tubular electrodes 602 and 604 and put tubular electrodes 602 and 604 in contact with margins of a dissected cancerous tumor. Exemplary sealed cover 608 was fabricated by a 3D printer.

### Example 2: Detecting Cancerous Status of Margins of Dissected Tumors from Mice

In this example, 10 female BALB/C mice that were 5 to 6 weeks old were tumorized with 4T1 cell line. 4T1 cell line is a mouse type breast cancer cell line with invasive phenotypes. 4T1 Cells were kept in DMEM culture medium complimented with 5% fetal bovine serum and 1% penicillin/streptomycin at 37° C. (5% CO₂, 95% filtered air). A manual cell counting method (i.e., haemocytometer neubauer) was used to determine primary populations of the cultured cell lines. 10 female BALB/C mice were tumorized by subcutaneously implanting of about 2×10⁶/0.2 ml⁻¹ 4T1-derived cancer cells into back of the 10 female BALB/C mice under 50 mg/kg of ketamine and 10 mg/kg of xylazine anesthesia. The 10 female BALB/C mice were maintained in individual groups with similar size of formed tumors with sharp histological distinct patterns. After about 14 days, exemplary method 300 was applied to normal and tumoral regions of dissected cancerous masses (with similar size and volume) from the 10 BALB/C mice models tumorized by 4T1 breast cancer cell lines utilizing exemplary system 102 using exemplary fabricated bioimpedance sensor 600. Electrical measurements were carried out by vacuum-assisted connection between electrodes 602 and 604 and mice tissues utilizing a rotary pump similar to vacuum pump 108.

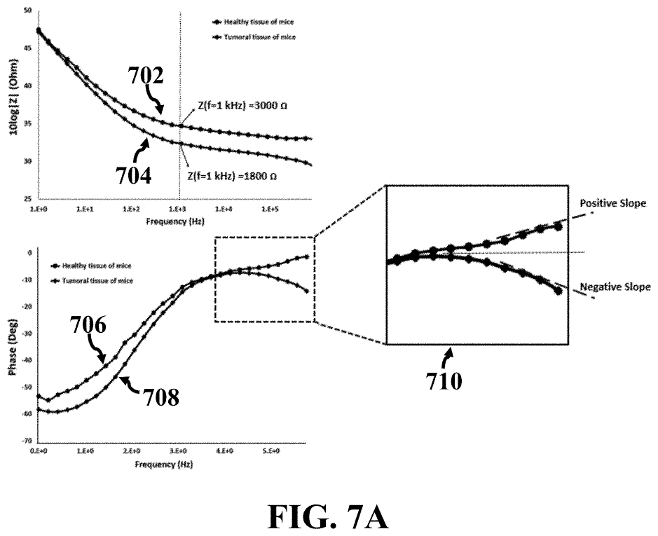

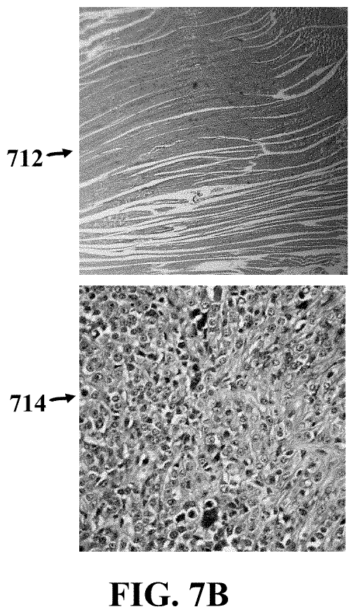

Impedance spectroscopy recorded from 35 frequency points in a ranges of 1 Hz to 1 MHz and voltage amplitude of 0.4 V revealed noticeable differences between normal and tumoral tissues. FIG. 7A shows impedance magnitude and phase diagrams of an exemplary normal muscle and an exemplary tumor tissue of mice recorded by exemplary system 100, consistent with one or more exemplary embodiments of the present disclosure. Normal muscular tissue and tumor tissue of mice were pathologically evaluated tumor and normal mice tissues. FIG. 7B shows exemplary H&E assays of exemplary mice healthy muscular tissue (image 712) and exemplary mice malignant tissue (image 714) respective to recorded impedance magnitude and phase diagrams of FIG. 7A, consistent with one or more exemplary embodiments of the present disclosure.

Referring to FIG. 7A, plots 702 and 704 in up panel represent impedance magnitude of normal muscle and tumor tissue, respectively. Impedance magnitude was investigated in f=1 kHz, where the diagram is almost going to be flattened. Plots 702 and 704 show a higher value of the Z_{1 kHz} for normal tissues than cancerous ones. Image 712 of FIG. 7B shows H&E assay result of a mice healthy muscular tissue which was recorded with Z_{1kKz}=3kΩ and IPS=3.6 utilizing exemplary system 100. Moreover, Image 714 of FIG. 7B shows H&E assay result of a mice malignant tissue with Z_{1 kHz}=1.8kΩ and IPS=−4. Several atypical cells with large nuclei and high (N/C) ratio may be observed in background of fibrotic tissue in H&E assay in image 714.

Referring more to FIG. 7A, plots 706 and 708 in bottom panel represent phase diagrams of normal muscle and tumor tissue, respectively. A distinct pattern of phase slope in a frequency range of 100 kHz to 500 kHz may be observable among different tissue types. A magnified view 710 of phase diagrams in frequency ranges greater than about 10 kHz shows a drastic drop for tumor tissue while a positive slope for normal tissue may be observed. This may lead to an extremum in phase diagram of mice tumor tissue below a frequency of about 100 kHz.

Impedance magnitudes and phases of mice muscles/non-tumoral tissues showed some significantly repeated specifications different from responses recorded from tumor tissues. A considerable increase in impedance magnitudes of normal tissues versus cancerous ones may be observable from the frequency of 1 kHz (plots 702 and 704 of FIG. 7A). Moreover, drastic changes in slope of the phase diagram (IPS) (plots 706 and 708 of FIG. 7A) near presence of an extremum in frequencies higher than 100 kHz was just observed in tumoral regions while normal lesions showed no extremum in their impedance phase spectrum in the same range of frequencies (magnified plot 710 of FIG. 7A).

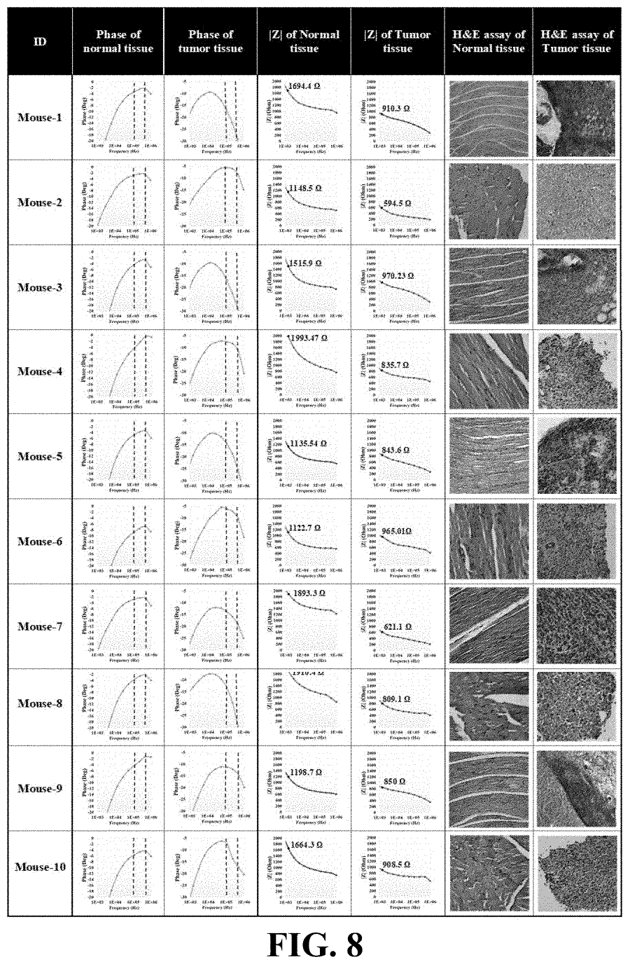

Hence, an impedance real value in 1 kHz (Z_{1 kHz}) and impedance phase slope in frequencies between 100 kHz and 500 kHz (IPS) were selected as targeted classification parameters for electrical characterization of different types of mice tissues. Z_{1 KHz} may reflect behavior of tissue in an alpha dispersion region. Contact impedance may be determinative less than this frequency and it makes faults on precise classification. Also, IPS may completely reflect dielectric activity of the tested tissue. FIG. 8 shows impedance spectroscopy of normal and cancerous regions tested from 10 mice models with their related Z_{1 kHz} and IPS in frequencies between 100 kHz and 500 kHz and H&E assays, consistent with one or more exemplary embodiments of the present disclosure. As illustrated in this figure, IPS for normal tissues may be a positive value and in contrast, IPS for cancerous tissues may be negative. Electrical phases of normal and tumor tissues are presented in the second and third columns of FIG. 8. Frequency ranges of 100 kHz to 500 kHz are marked by black dashed lines in the diagram. A trend of the phase values for normal tissue is increasing, while it is decreasing for tumor tissue. It means that IPS is positive in normal tissues while it is negative for cancerous tissues. The 4^{th} and 5^{th} columns include illustrations of impedance magnitudes of normal and malignant tissues, respectively. Z_{1 kHz} of cancer tissues were higher than that in normal ones. It may be noticeable that drastic changes in slope of phase diagram near the presence of an extremum in frequencies higher than 100 kHz were just observed in tumoral regions while normal lesions showed no extremum in their high-frequency impedance phase response. Also, it can be inferred from magnitude diagrams and Z_{1 kHz} values of FIG. 8 that mice cancerous tissues may have lower impedance magnitudes than mice normal muscular tissues. Images of pathological H&E assay of each type of tissue was inserted in the last columns of FIG. 8. It may be seen that the size and number of cancerous cells (which have atypical vesicular nuclei) in tumor tissues are more than muscular tissues.

### Example 3: Detecting Cancerous Status of Margins of Human Dissected Tumors

In this example, an exemplary method similar to method 300 utilizing exemplary fabricated bioimpedance sensor 600 and system 100 was applied to fresh margin tissues dissected from breast cancer patients which have been sent for intraoperative frozen-section. Impedance spectroscopy of 313 different samples (e.g., cancerous tumors, benign lesions, fibro-fatty tissues, neo-adjuvant mastectomy cases, etc.) obtained from surface of masses had been dissected from 68 patients accomplished in a frequency range of 1 Hz to 1 MHz.

After preparing touch imprint cytology slides from dissected breast tumor masses (first step of the intraoperative frozen-section in pathology labs), margins were pre-evaluated by a pathologist. Then, steps of exemplary method 300 was applied on tumor margins, before the main frozen-section process, to record an impedance spectroscopy of superficial surface all around an exemplary dissected breast tumor mass without making a dissection or intervention on the tumor mass. This process may be done for suspicious regions for a pathologist on margins of an exemplary dissected breast tumor. Then, a pathologist cut those suspicious margins to do intraoperative frozen-section diagnosis including mounting a tissue on a cryostat stage for freezing, fast fixing, sectioning, and hematoxylin-eosin (H&E) staining. After evaluation of the frozen slides by a pathologist, margin diagnostic results was declared. Samples were then sent for formalin based fixation and permanent pathology procedure.

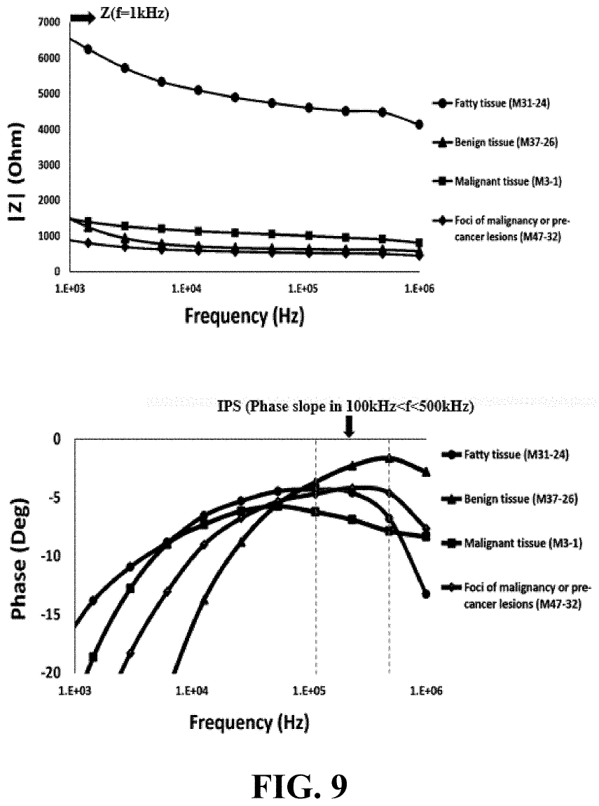

Different types of histological patterns with different ranges of impedance spectroscopy responses (Z_{1 kHz} and IPS) were observed in human breast tissue during the tests. FIG. 9 shows impedance magnitude and phase diagrams for different types of tissues (with pathologically distinct patterns) based on the measured Z_{1 kHz} and IPS, consistent with one or more exemplary embodiments of the present disclosure. Each type of tissue showed different patterns of impedance and phase which were helpful in classification of impedance spectroscopy responses which may further utilized for detecting cancerous status of an unknown margin of a dissected breast tumor. Some repeatable observations may be noticeable in the electrical recording of tissues. In tissues containing glandular cells (ducts and lobules independent from the presence of malignancy or not), Z_{1 kHz} may be much lower than fatty tissues. Also, IPS values of tissues containing glandular cells may be more than about 0.3 in almost all benign lesions, while it may be negative in malignant tissues. Although most fatty tissues show higher negative IPS than malignant ones, Z_{1 kHz} of fatty lesions distinct them from other types of tissues. Hence, independent evaluating of IPS in fatty tissues may be not required.

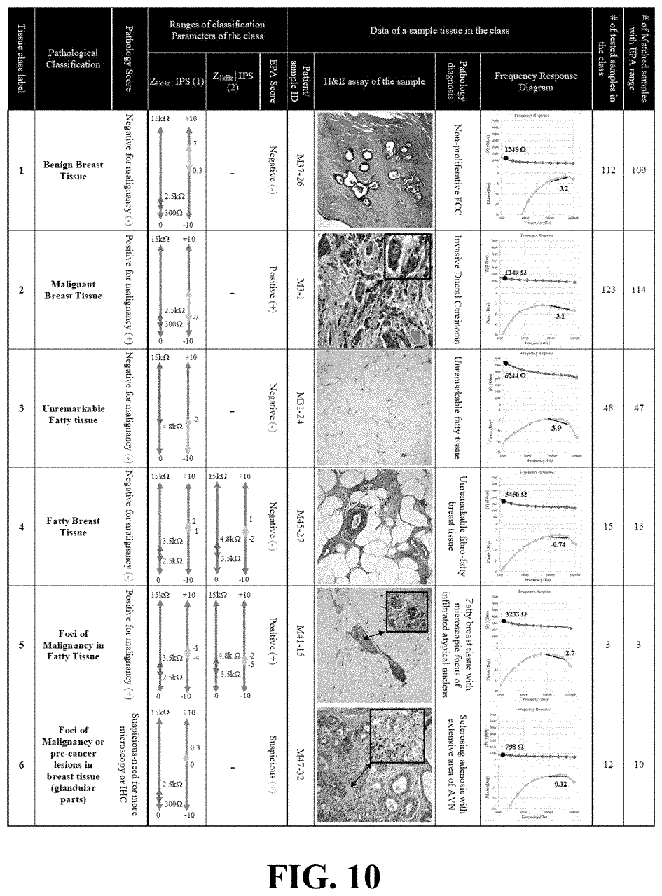

Based on these findings, comparative analyses were carried out on tested samples and six different types of tissues were classified in impedance spectroscopy scoring due to co-analyzing of Z_{1 kHz} and IPS results of tested samples. Such classified parameters showed well-matching with pathological classification in intraoperative frozen-section. FIG. 10 shows six types of breast tissues and their respective impedance spectroscopy classification parameters with an example for each type, consistent with one or more exemplary embodiments of the present disclosure. H&E assay, permanent pathological results, impedance magnitude diagram, impedance phase diagram, and classification parameters for each example are included in FIG. 10. These six types of tissues are listed below:

Type 1 includes benign breast lesions including non-proliferating fibrocystic changes (FCC), columnar cell changes (CCC), columnar cell hyperplasia (CCH), usual ductal hyperplasia (UDH), terminal ductal lobular unit (TDLU), fibroadenoma, etc. with Z_{1 kHz} less than 2.5 kΩ and IPS value more than 0.3 (e.g., sample ID M37-26 in FIG. 10 that includes non-proliferative FCC with unremarkable glandular epithelial cells in background of connective tissue may be observed, where Z_{1 kHz} and IPS are 1.2 kΩ and 3.2, respectively).

Type 2 includes pre-malignant/malignant lesions with extensive distribution among stroma such as invasive ductal carcinoma (IDC), invasive lobular carcinoma (ILC), ductal carcinoma in-situ (DCIS), lobular carcinoma in-situ (LCIS), atypical ductal hyperplasia (ADH), atypical lobular hyperplasia (ALH), etc. with Z_{1 kHz} less than 2.5 kΩ and negative values of IPS (e.g., sample ID M3-1 in FIG. 10 that includes invasive ductal carcinoma with atypical epithelial cells and high nucleus to cytoplasm size ratio may be observed, where Z_{1 kHz} and IPS are 1.25 kΩ and −3.1, respectively).

Type 3 includes fatty tissues with Z_{1 kHz} more than 4.8 kΩ and negative values of IPS (e.g., sample ID M31-24 in FIG. 10 with Z_{1 kHz} and IPS are 6.25 kΩ and −3.9, respectively may be observed).

Type 4 includes breast glandular tissues that contain some fatty components (named as fatty breast tissues). Two classification parameter ranges were defined for this group. First, 2.5 kΩ<Z_{1 kHz}<3.5 kΩ with IPS between −1 and 2 and second, 3.5kΩ<Z_{1 kHz}<4.8 kΩ with IPS between −2 and 1 may be observed (e.g., sample ID M45-27 in FIG. 10 with Z_{1 kHz} and IPS 3.47 kΩ and −0.74, respectively).

Type 5 includes Foci of pre-malignant/malignant lesions among fatty tissue. Two classification parameter ranges were defined for this group. First, 2.5 kΩ<Z_{1 kHz}<3.5 kΩ with IPS between −4 and −1 and second, 3.5kΩ<Z_{1 kHz}<4.8 kΩ with IPS between −5 and −2 (e.g., sample ID M41-15 in FIG. 10 that includes fatty breast tissue with a microscopic focus of infiltrated atypical nucleus with Z_{1 kHz} and IPS are 3.2 kΩ and −2.7, respectively).

Type 6 includes Foci of pre-malignant/malignant lesions among benign breast tissue with Z_{1 kHz} less than 2.5 kΩ and IPS values between 0 more 0.3 (e.g., sample ID M47-32 in FIG. 10 that includes Sclerosing adenosis with an extensive area of atypical vesicular nuclei with Z_{1 kHz} and IPS of 0.7 kΩ and 0.12, respectively).

Considering class labels 2, 5, and 6 as positive scores and class labels 1, 3, and 4 as negative scores of cancer, an accuracy of correlated impedance spectroscopy-pathology classification was about 90% in 313 tested margin samples. This calibration by classifying cancerous status of margins of a dissected breast tumor into above-mentioned 6 groups was suggested by considering all of the 313 tested samples. So, it may be sufficient to use only 3 frequency points (1 kHz, and 100 kHz and 500 kHz) to extract electro-pathological classification parameters for identifying cancerous status of margins of a freshly dissected tumor.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1- A method for identifying cancerous status of margins of a tumor, comprising:
measuring two impedimetric criteria associated with a target region of surface of a tumor tissue dissected less than 30 minutes from a human or an animal, comprising:
measuring an electrical impedance magnitude of the target region at a frequency of 1 kHz (Z_{1 kHz}); and
measuring an impedance phase slope (IPS) of the target region in a frequency range of 100 kHz to 500 kHz, comprising:
measuring a first electrical impedance phase value (Phase₁) at a first frequency value of Frequency₁ equal to 100 kHz;
measuring a second electrical impedance phase value (Phase₂) at a second frequency value of Frequency₂ equal to 500 kHz; and
calculating the IPS being defined by:
$$IPS = \frac{{Phase}_{2} - {Phase}_{1}}{log ( {Frequency}_{2} ) - log ( {Frequency}_{1} )} ;$$
and
detecting a cancerous status of the target region, comprising:
determining the target region being a benign region responsive to the measured Z_{1 kHz} being less than a first reference impedance value and the measured IPS being more than a first reference IPS;
determining the target region being a cancerous region responsive to the measured Z_{1 kHz} being less than the first reference impedance value and the measured IPS being less than a second reference IPS, the second reference IPS being equal to the first reference IPS or less; and
determining the target region being a fatty region responsive to the measured Z_{1 kHz} being more than a second reference impedance value.

2- A method for identifying cancerous status of margins of a tumor, comprising:
putting two electrodes of a bioimpedance sensor in contact with a target region of surface of a freshly dissected tumor tissue;
measuring two impedimetric criteria associated with the target region, comprising:
measuring an electrical impedance magnitude of the target region at a frequency of 1 kHz (Z_{1 kHz}); and
measuring impedance phase slope (IPS) of the target region in a frequency range of 100 kHz to 500 kHz; and
detecting a cancerous status of the target region, comprising:
determining the target region being a benign region responsive to the measured Z_{1 kHz} being less than a first reference impedance value and the measured IPS being more than a first reference IPS;
determining the target region being a cancerous region responsive to the measured Z_{1 kHz} being less than the first reference impedance value and the measured IPS being less than a second reference IPS, the second reference IPS being equal to the first reference IPS or less; and
determining the target region being a fatty region responsive to the measured Z_{1 kHz} being more than a second reference impedance value.

3- The method of claim 2, wherein measuring the two impedimetric criteria associated with the target region comprises:
connecting the two electrodes of the bioimpedance sensor to an impedance analyzer device;
applying an alternating current (AC) voltage in a sweeping range of frequencies to the two electrodes, the sweeping range of frequencies comprising a frequency range between 1 kHz and 500 kHz;
measuring an impedance magnitude of an electrical impedance value of the target region at frequency of 1 kHz (Z_{1 kHz});
measuring a set of electrical impedance phase values respective to the swept range of frequencies between 100 kHz and 500 kHz; and
calculating the IPS respective to the swept range of frequencies between 100 kHz and 500 kHz.

4- The method of claim 3, wherein applying the AC voltage in the sweeping range of frequencies to the two electrodes comprises applying an AC voltage with an amplitude of 0.4 V in the sweeping range of frequencies to the two electrodes.

5- The method of claim 3, wherein calculating the IPS comprises calculating a slope of the measured set of electrical impedance phase values versus the swept range of frequencies defined by:
$$IPS = \frac{{Phase}_{2} - {Phase}_{1}}{log ( {Frequency}_{2} ) - log ( {Frequency}_{1} )} ,$$
Wherein Phase₁ is a measured impedance phase value at a first frequency value of Frequency₁ of 100 kHz and Phase₂ is a measured impedance phase value at a second frequency value of Frequency₂ of 500 kHz.

6- The method of claim 2, wherein the freshly dissected tumor tissue comprises a tumor tissue dissected less than 30 minutes from a human or an animal.

7- The method of claim 2, wherein the target region comprises a part of surface of the freshly dissected tumor tissue with an area of 4 mm² and a depth of 2 mm.

8- The method of claim 2, wherein putting the two electrodes of a bioimpedance sensor in contact with the target region comprises putting two respective distal ends of the two electrodes on surface of the freshly dissected tumor tissue at the target region.

9- The method of claim 8, wherein putting the two electrodes of a bioimpedance sensor in contact with the target region further comprises generating a uniform pressurized contact between the respective distal ends of the two electrodes and the target region by applying a vacuum suction pressure throughout the two electrodes, comprising:
connecting a vacuum pump to respective proximal ends of the two electrodes utilizing a tubular line; and
applying a vacuum pressure of at least 20 KPa to the respective proximal ends of the two electrodes utilizing the vacuum pump.

10- The method of claim 2, wherein detecting the cancerous status of the target region comprises detecting the cancerous status of a target region of surface of a freshly dissected breast tumor, comprising:
determining the target region being a benign breast region responsive to the measured Z_{1 kHz} being less than 2.5 kΩ and the measured IPS being more than 0.3;
determining the target region being a cancerous breast region responsive to the measured Z_{1 kHz} being less than 2.5 kΩ and the measured IPS being negative (less than zero); and
determining the target region being a fatty breast region responsive to the measured Z_{1 kHz} being more than 4.8 kΩ.

11- The method of claim 10, wherein detecting the cancerous status of the target region further comprises:
determining the target region being a benign breast region having clusters of cancerous breast cells responsive to the measured Z_{1 kHz} being less than 2.5 kΩ and the measured IPS being between zero and 0.3;
determining the target region being a benign fatty breast region responsive to a range for the measured Z_{1 kHz} and the measured IPS comprising at least one of:
the measured Z_{1 kHz} being between 2.5 kΩ and 3.5 kΩ and the measured IPS being between −1 and 2; and
the measured Z_{1 kHz} being between 3.5 kΩ and 4.8 kΩ and the measured IPS being between −2 and 1; and
determining the target region being a fatty breast region having clusters of cancerous breast cells responsive to a range for the measured Z_{1 kHz} and the measured IPS comprising at least one of:
the measured Z_{1 kHz} being between 2.5 kΩ and 3.5 kΩ and the measured IPS being between −4 and −1; and
the measured Z_{1 kHz} being between 3.5 kΩ and 4.8 kΩ and the measured IPS being between −5 and −2.

12- A system for identifying cancerous status of margins of a tumor, comprising:
a bioimpedance sensor comprising:
at least two tubular electrodes, each respective electrode of the two tubular electrodes comprising an electrically conductive hollow rod, each respective electrode comprising a distal end and a proximal end, each respective distal end configured to be put in contact with a target region of surface of a tumor tissue dissected less than 30 minutes from a human or an animal, each respective proximal end configured to be connected to an impedance analyzer device;
an impedance analyzer device, the impedance analyzer device being in connection with respective proximal ends of the at least two tubular electrodes via at least one of an electrical connector and a wireless connection; and
a processing unit electrically connected to the impedance analyzer device, the processing unit comprising:
a memory having processor-readable instructions stored therein; and
a processor configured to access the memory and execute the processor-readable instructions, which, when executed by the processor configures the processor to perform a method, the method comprising:
applying, utilizing the impedance analyzer device, an alternating current (AC) voltage in a sweeping range of frequencies to the at least two tubular electrodes, the sweeping range of frequencies comprising a frequency range between 1 kHz and 500 kHz;
measuring, utilizing the impedance analyzer device, an electrical impedance value of the target region at frequency of 1 kHz (Z_{1 kHz});
measuring, utilizing the impedance analyzer device, a set of electrical impedance phase values respective to the swept range of frequencies between 100 kHz and 500 kHz;
calculating impedance phase slope (IPS) respective to the swept range of frequencies between 100 kHz and 500 kHz; and
detecting cancerous status of the target region based on the measured Z_{1 kHz} and the calculated IPS.

13- The system of claim 12, wherein detecting cancerous status of the target region based on the measured Z_{1 kHz} and the calculated IPS comprises:
determining the target region being a benign region responsive to the measured Z_{1 kHz} being less than a first reference impedance value and the measured IPS being more than a first reference IPS;
determining the target region being a cancerous region responsive to the measured Z_{1 kHz} being less than the first reference impedance value and the measured IPS being less than a second reference IPS, the second reference IPS being equal to the first reference IPS or less; and
determining the target region being a fatty region responsive to the measured Z_{1 kHz} being more than a second reference impedance value.

14- The system of claim 13, wherein:
the freshly dissected tumor tissue comprises a dissected breast tumor, and
detecting the cancerous status of the target region comprises:
determining the target region being a benign breast region responsive to the measured Z_{1 kHz} being less than 2.5 kΩ and the measured IPS being more than 0.3;
determining the target region being a cancerous breast region responsive to the measured Z_{1 kHz} being less than 2.5 kΩ and the measured IPS being negative (less than zero); and
determining the target region being a fatty breast region responsive to the measured Z_{1 kHz} being more than 4.8 kΩ.

15- The system of claim 14, wherein detecting the cancerous status of the target region further comprises:
determining the target region being a benign breast region having clusters cancerous breast cells responsive to the measured Z_{1 kHz} being less than 2.5 kΩ and the measured IPS being between zero and 0.3;
determining the target region being a benign fatty breast region comprising a plurality of fatty breast cells responsive to a range for the measured Z_{1 kHz} and the measured IPS comprising at least one of:
the measured Z_{1 kHz} being between 2.5 kΩ and 3.5 kΩ and the measured IPS being between −1 and 2; and
the measured Z_{1 kHz} being between 3.5 kΩ and 4.8 kΩ and the measured IPS being between −2 and 1; and
determining the target region being a fatty breast region having clusters of cancerous breast cells responsive to a range for the measured Z_{1 kHz} and the measured IPS comprising at least one of:
the measured Z_{1 kHz} being between 2.5 kΩ and 3.5 kΩ and the measured IPS being between −4 and −1; and
the measured Z_{1 kHz} being between 3.5 kΩ and 4.8 kΩ and the measured IPS being between −5 and −2.

16- The system of claim 12, wherein calculating the IPS comprises calculating a slope of the measured set of electrical impedance phase values versus the swept range of frequencies defined by:
$$IPS = \frac{{Phase}_{2} - {Phase}_{1}}{log ( {Frequency}_{2} ) - log ( {Frequency}_{1} )} ,$$
Wherein Phase₁ is a measured impedance phase value at a first frequency value (Frequency₁) of 100 kHz and Phase₂ is a measured impedance phase value at a second frequency value (Frequency₂) of 500 kHz.

17- The system of claim 12, wherein:
the system further comprises a vacuum pump configured to:
be connected to the respective proximal ends of the at least two tubular electrodes utilizing a tubular line; and
be electrically connected to the processing unit, and
the method further comprising:
forming a uniform connection between distal ends of the at least two tubular electrodes and the target region by applying, utilizing the vacuum pump, a vacuum pressure of at least 20 KPa to the respective proximal ends of the at least two tubular electrodes.

18- The system of claim 12, wherein:
each respective electrode of the two tubular electrodes comprises a stainless steel hollow rod with a length between 10 mm and 20 mm and an internal diameter between 0.5 mm and 2 mm, and
an electrically insulating layer with a thickness between 0.5 mm and 1 mm placed around parts of each respective electrode of the two tubular electrodes.

19- The system of claim 12, wherein the bioimpedance sensor further comprises:
an electrode holder comprising at least two hollow openings, each hollow opening of the at least two hollow openings encompassing a middle part of each electrode of the at least two tubular electrodes, the middle part of each electrode comprising a respective part of each electrode except the respective distal end and the proximal end;
a handle comprising a tubular member, the handle comprising a distal end and a proximal end, the electrode holder fixed inside the distal end, the handle configured to facilitate utilizing the at least two tubular electrodes, comprising:
facilitate putting the respective distal ends of the at least two tubular electrodes with the target region;
facilitate applying a vacuum pressure through the at least two tubular electrodes; and
contain an electrical wire connecting the impedance analyzer device to the respective proximal ends of the at least two tubular electrodes; and
a cap configured to seal the proximal end of the handle by fastening the cap around the proximal end, the cap comprising two openings comprising:
a first opening configured to pass the electrical wire there through, the electrical wire connected to the impedance analyzer device; and
a second opening configured to connect to a vacuum pump by fastening a flexible tubular line around the second opening, the flexible tubular line connected to the vacuum pump.

20- The system of claim 12, wherein each two respective openings of the at least two hollow openings embedded on the electrode holder has a distance between 2 mm and 5 mm.
